(19) United States
(12) Patent Application Publication
(10) Pub. No.: US 2022/0323258 A1
(43) Pub. Date: Oct. 13, 2022

(54) EAR SHAPING DEVICE

(71) Applicant: Elayne Sheridan, Awendaw, SC (US)

(72) Inventor: Elayne Sheridan, Awendaw, SC (US)

(21) Appl. No.: 17/717,109

(22) Filed: Apr. 10, 2022

Related U.S. Application Data
(60) Provisional application No. 63/173,693, filed on Apr. 12, 2021.

(51) Int. Cl. A61F 11/00; A61F 5/01; A44C 15/00

(52) U.S. Cl. CPC A61F 11/00; A61F 5/01; A44C 15/0005

(57) ABSTRACT

The ear shaping device is designed to assist a user in the temporary correction of protruding ears. More particularly, the device provides the user with many options and potential adjustments to correct the user's protruding ear to his or her individual specification.

### CROSS-REFERENCE TO RELATED APPLICATIONS

This application claims the benefit of U.S. Provisional Application No. 63/173,693, filed on Apr. 12, 2021, which is hereby incorporated in its entirety.

### FIELD OF THE INVENTION

The present invention is directed to an ear shaping device designed to assist a user in the temporary correction of protruding ears by several centimeters. More particularly, the invention provides the user with many options and potential adjustments to correct the user's protruding ear to his or her individual specification.

### BACKGROUND OF THE INVENTION

The following description is not an admission that any of the information provided herein is prior art or relevant to the present invention, or that any publication specifically or implicitly referenced is prior art. Any publications cited in this description are incorporated by reference herein. Where a definition or use of a term in an incorporated reference is inconsistent or contrary to the definition of that term provided herein, the definition of that term provided herein applies and the definition of that term in the reference does not apply.

In many Western cultures, protruding ears can be a cause of ridicule leading to embarrassment, feeling insecure, and can have a negative effect on a person's quality of life. In a study on adolescents with behavior problems, 40% of those adolescents were noted with having an external-ear deformity. Matthew B. Zavod et al., Complications in Head and Neck Surgery, Complications of Otoplasty 769 (David W. Eisele et al., ed., 2009). Lifelong feelings of insecurity and inadequacy associated with having protruding ears can negatively impact academic and career success. Thus, the ability to correct an external ear deformity is essential.

Currently, there are surgical and non-surgical options for people who seek to permanently reposition or reshape his or her protruding ear. These medical interventions are highly recommended for children but may also be effective for adults. However, these medical interventions may require reconstructive surgery, sleeping for several months with a fitted device attached to ears, or implanting a shaped corrective wire under the skin. These interventions vary in cost, effectiveness, risk of complications or scarring, and inconvenience. The costs for these types of medical interventions can range from hundreds to thousands of dollars. Currently, there are cosmetic solutions for individuals who prefer a nonsurgical option. One approach is to apply tape to the back of the ear and side of the head (e.g., U.S. Pat. No. 5,076,262). In another approach, a user can clip ear to strands of his or her hair (e.g. U.S. Pat. No. 4,187,838). In yet another approach, a user wraps a tension band around the back or top of the head, while the bands draw against clamps attached to the ears. However, all of these approaches fall short of meeting the need for a durable, comfortable, attractive, and effective cosmetic solution.

A bar earring exits in the prior art that was designed to compress the thickness of the helix (the rim of the upper ear). However, the device does not appear to significantly affect the shape of the antihelix (the flat portion of the upper ear). In addition, the bar earring is only effective for 40% of users. Lot Ear Cuffs, https://www.etsy.com/listing/934854560/new-ear-cuff-for-protruding-ears-no?ref=shop home active 3 (last visited Feb. 28, 2021).

There exists an ear cuff that encircles the antihelix. However, this device creates an obvious and unattractive fold in the upper portion of the antihelix.

Thus, there exists a need for an ear shaping device that is inexpensive, customizable, comfortable, and attractive to wear that can temporarily reduce the amount of protrusion of a user's prominent ears.

### SUMMARY OF THE INVENTION

The present invention is directed to an ear shaping device designed to assist a user in the temporary correction of protruding ears by several centimeters. More particularly, the invention provides the user with many options and potential adjustments to correct the user's protruding ear to his or her individual specification. The device comprises a top portion, a bottom portion, an interior surface, an exterior surface, and an arc with an adjustable angle. In one embodiment, the material of construction is sterling silver. The curve of the top portion and bottom portion are configured to enable the user to reduce the amount of protrusion of his or her ear by creating a seesaw movement whereby force from the bottom portion of the device on the cymba results in the top of the ear moving closer to the user's head.

In one embodiment, the device is a single piece construction designed with an arc angle of about 112° resulting in a reduction of a protruding ear of about 2 cm.

In another embodiment, the device comprises a top portion, a bottom portion, an interior surface, an exterior surface, an arc with an adjustable angle, and a decorative element. In this embodiment, the decorative element is permanently affixed to the exterior surface to provide the user with an added aesthetically pleasing look. Decorative elements may include engravings, stampings, patinas, beads, crystals, or gemstones or any combination thereof.

In another embodiment, the device comprises a top portion, a bottom portion, an interior surface, an exterior surface, an arc with an adjustable angle, and a decorative element. The decorative element is removably affixed to the exterior surface. In this embodiment, the user can change the decorative element to provide the user with various aesthetically pleasing options.

In yet another embodiment, the device comprises a top portion, a bottom portion, an interior surface, an exterior surface, an arc with an adjustable angle, a decorative element and one or more legs. In this embodiment, the top portion comprises two legs that is configured to enable the device to slide over the helix to further promote the correction of the user's ear protrusion. In yet another embodiment, the top portion comprises one leg that is configured to enable the device to slide over the helix.

In yet another embodiment, the device comprises a top portion, a bottom portion, an interior surface, an exterior surface, an arc with an adjustable angle, and a middle portion comprised of a plurality of beads and a plurality of rings. The device comprises a single piece construction in the shape of a forward slanted “C” wherein the middle portion comprises a plurality of beads and rings permanently affixed to each other in an alternating pattern. Furthermore, the material of construction for the rings is a malleable metal enabling the user to stretch the rings resulting in a longer middle portion to accommodate a larger ear. The top portion of the device extends along the back of the superior crus of antihelix while the bottom portion extends from the front of the superior crus of antihelix to the cymba allowing the middle portion to rest on the user's scapha and antihelix.

The material of construction for the device includes resin, plastic, any precious metal, any base metal, any reactive metal, sheet metal, wire, wire mesh, or wood, and or any combination thereof. The device can be constructed by using casting, molding, die cutting and forming, wire forming, metalsmithing, or 3-dimensional printing. Furthermore, interior coatings or silicone padding may be attached to the device to add to the user's comfort.

Various objects, features, aspects and advantages of the inventive subject matter will become more apparent from the following detailed description of exemplary embodiments, along with the accompanying figures in which like numerals represent like components.

### BRIEF DESCRIPTION OF THE DRAWINGS

FIG. 1 depicts a perspective view of an exemplary embodiment of the ear shaping device.

FIG. 2 depicts a perspective view of an exemplary embodiment of the ear shaping device.

FIG. 3 depicts the front view of an exemplary embodiment of the ear shaping device.

FIG. 4 depicts the perspective view of an alternate embodiment of the ear shaping device comprising a decorative element.

FIG. 5 depicts the side view of an alternate embodiment of the ear shaping device.

FIG. 6 depicts the side view of an alternate embodiment of the ear shaping device comprising a decorative element and a leg.

FIG. 7 depicts the perspective view of an alternate embodiment of the ear shaping device comprising a plurality of rings and beads.

FIG. 8 depicts the perspective view of an alternate embodiment of the ear shaping device comprising a ring.

FIG. 9 depicts the perspective view of an alternate embodiment of the ear shaping device comprising a stretched ring.

FIG. 10 depicts the front perspective view of the exemplary embodiment of the ear shaping device on a user's ear.

FIG. 11 depicts the back perspective view of the exemplary embodiment of the ear shaping device on a user's ear.

### DETAILED DESCRIPTION

The present invention is directed to an ear shaping device that can be used on either ear to temporarily depress the shape of a protruding ear. The figures depict exemplary embodiments of device for the user's left ear. The device fitted for the right ear is a mirror image of the above figures. In particular, the device comprises a customizable single piece made from durable material wherein the angle in the arc of the earring and the curve of the bottom portion in relation to the curve of the top portion are configured to bring the user's ear to lay closer to the side of the user's head, thus, minimizing the appearance of the user's protruding ears.

In an exemplary embodiment, the device comprises an arc with a varying angle, a top portion, a bottom portion, an interior surface, and an exterior surface to temporarily correct a user's protruding ear. The device takes the shape of a forward-slanted “C” and the user slides the device over the helix located at the top of the ear whereby the interior surface of the device is touching the back of the user's ear.

The length of the device can range from about 0.5″ to 1.75″ and the width of the device can range from about 0.375″ to 0.75″. In one embodiment, the thickness of the device ranges from about 0.9 mm to 1.5 mm. And in an embodiment, the angle of the arc ranges from 90° to 120° and can be adjusted based on the size and specific amount of protrusion of the user's ear.

In one embodiment, the material of construction of the device is silver or gold. In other embodiment, materials of construction include resin, plastic, any precious metal, any base metal, any reactive metal, sheet metal, wire, wire mesh, or wood, and or any combination thereof. Furthermore, a decorative element may be added to the device for aesthetic purposes in accordance with the user's preference. In addition, the comfort of the device may be enhanced with an interior coating or padding.

As used herein, and unless the context dictates otherwise, the terms “Ear Shaping Device,” “Ear Correcting Device,” and “device” are intended to represent the present invention and the terms are used interchangeably.

As used in the description herein and throughout the claims that follow, the meaning of “a,” “an,” and “the” includes plural reference unless the context clearly dictates otherwise. Also, as used in the description herein, the meaning of “in” includes “into” and “on” unless the context clearly dictates otherwise.

As used herein, the term “about” in conjunction with a numeral refers to a range of that numeral starting from 10% below the absolute of the numeral to 10% above the absolute of the numeral, inclusive.

As used in the description herein and throughout the claims that follow, the meaning of “cymba,” “superior crus of antihelix,” “helix,” “triangular fossa,” “inferior crus of antihelix,” “scapha,” and “antihelix” refers to their ordinary anatomical meanings of the ear. A “cymba” is defined as the upper part of the concha of the ear. A “superior crus of antihelix” is defined as the upper cartilaginous ridge arising at the bifurcation of the antihelix that separates the scapha from the triangular fossa. A “helix” is defined as the folded rim of skin and cartilage around the outer ear. A “triangular fossa” is defined as the concavity bounded by the superior and inferior crura of the antihelix and the ascending portion of the helix. An “inferior crus of antihelix” is defined as the lower cartilaginous ridge arising at the bifurcation of the antihelix that ends beneath the fold of the ascending helix. A “scapha” is defined as the groove between the helix and the antihelix. An “antihelix” is defined as the curved elevation of cartilage within or in front of the helix.

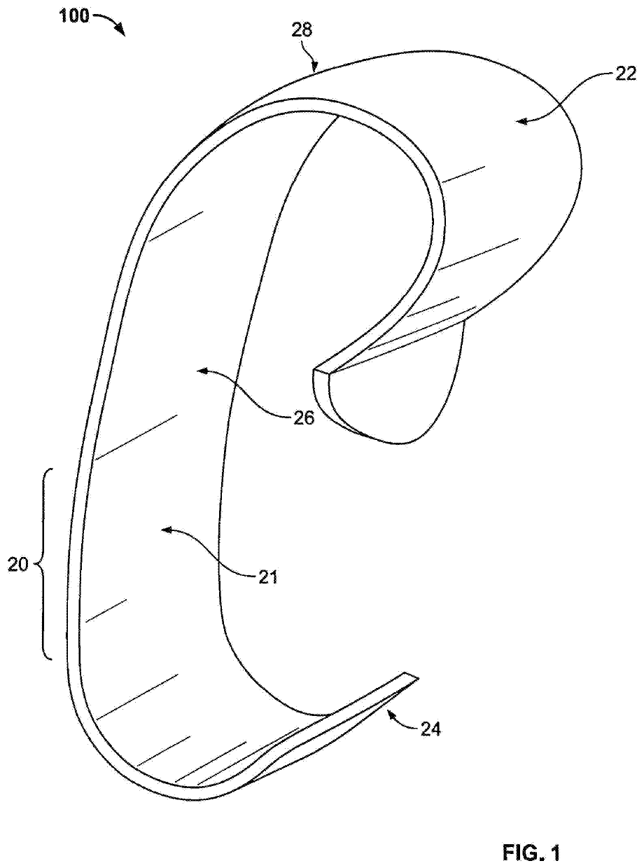

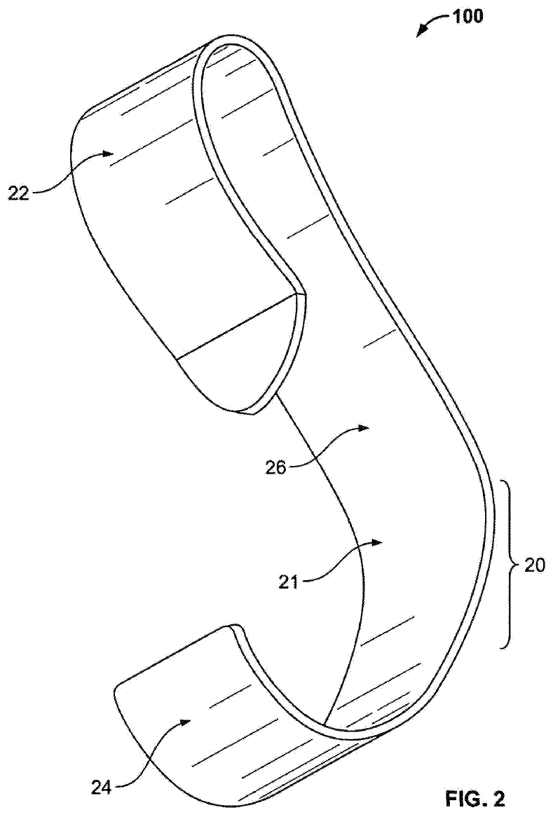

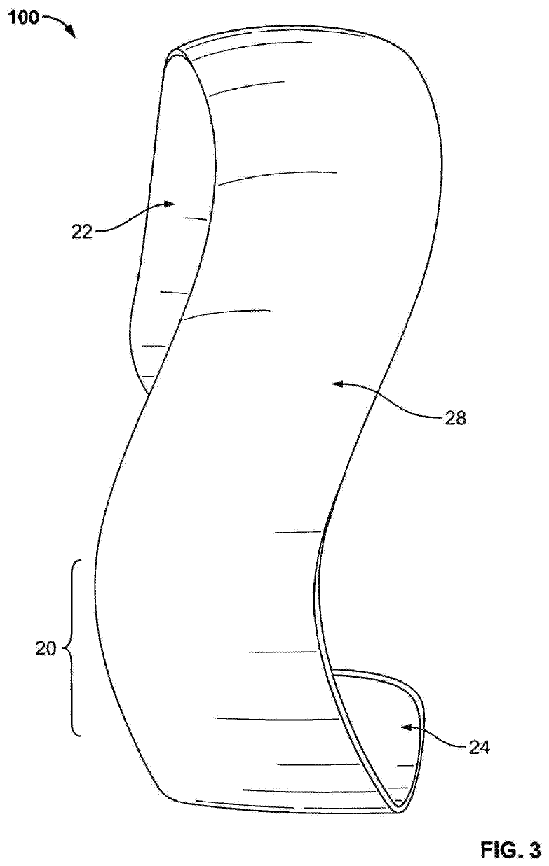

Exemplary embodiments of the present invention are depicted in FIGS. 1-3. In one embodiment, ear shaping device 100 comprises arc 20 with angle 21, top portion 22, bottom portion 24, interior surface 26, and exterior surface 28 (see FIG. 1). In one embodiment, device 100 is a single-piece construction, wherein top portion 22 and bottom portion 24 are curved inward towards interior surface 26 and top portion 22 has a greater curve compared to bottom portion 24. The shape of device 100 is configured such that it allows device 100 to be easily placed over a user's ear. In this embodiment, angle 21 of arc 20 ranges from about 90° to about 120° depending on the size and type of the user's protruding ear. In an exemplary embodiment, width of device 100 is about 0.75″ at its widest point and top portion 22 is about 0.375″ making device 100 about 1.25″ in length. In this embodiment, angle 21 is about 110°. In an embodiment, thickness of device 100 ranges from about 0.9 mm to 1.5 mm depending on the size and type of the user protruding ear.

In one embodiment, interior surface 26 extends across the user's ear from the front of the superior crus of antihelix to the cymba, and exterior surface 28 extends behind the user's ear along the back of the superior crus of antihelix wherein edge of bottom portion 24 presses against the cymba thereby forcing the top of the user's ear to lie closer to the side of the user's head.

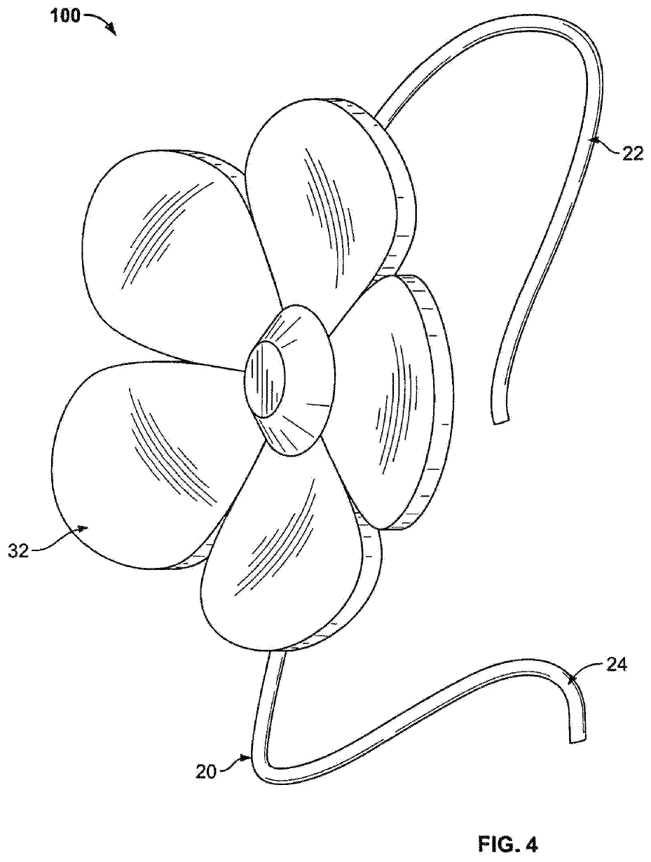

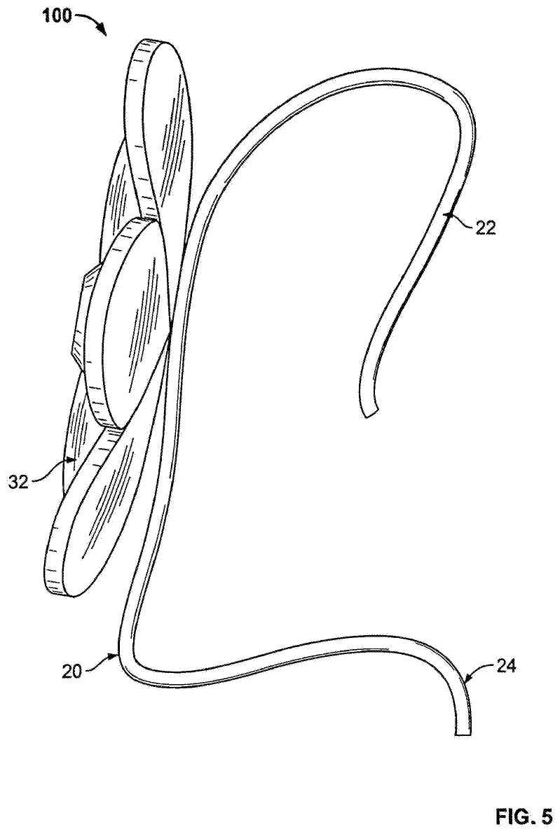

In yet another embodiment, device 100 comprises arc 20 with angle 21, top portion 22, bottom portion 24, interior surface 26, exterior surface 28, and decorative element 32 (see FIGS. 4-5). In this embodiment, device 100 is about 0.875″ in length, 0.375″ in width (not including the width of decorative element 32), top portion 22 is about 0.5″ and curved, and arc 20 comprises angle 21 at about 90° (angle not shown). In this embodiment, decorative element 32 comprises a gemstone or other embellishment and is permanently affixed to exterior surface 28. In this embodiment, top portion 22 extends over the user's helix and rests between the triangular fossa and the inferior crus of antihelix.

In yet another embodiment, decorative element 32 is removably affixed to exterior surface 28.

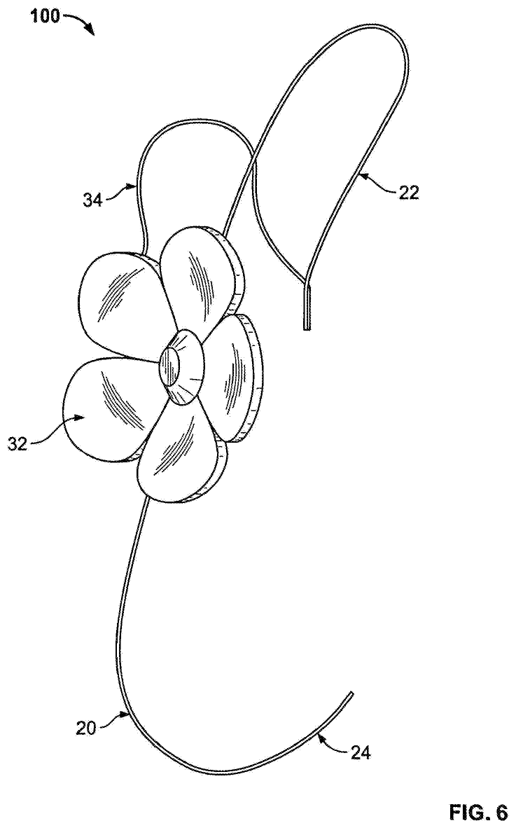

In yet another embodiment, ear shaping device 100 comprises arc 20 with angle 21, top portion 22, bottom portion 24, interior surface 26, exterior surface 28, decorative element 32, and leg 34 (see FIG. 6). In this embodiment, device 100 is a single-piece construction wherein top portion 22 and bottom portion 24 are curved inward towards interior surface 26 and top portion 22 has a greater curve compared to bottom portion 24. In an embodiment, angle 21 of arc 20 ranges from about 90° to about 120° depending on the type of the user's protruding ear. In this embodiment, leg 34 is permanently affixed to exterior surface 28 of top portion 22 and decorative element 32. In an alternative embodiment, leg 34 is permanently affixed to interior surface 26 of top portion 22 and decorative element 32.

In this embodiment, leg 34 extends over the user's ear at the helix while interior surface 26 extends across the user's ear from the front of the superior crus of antihelix to the cymba, and exterior surface 28 extends behind the user's ear along the back of the superior crus of antihelix. In this embodiment, top portion 22 is about 0.375″ making device 100 about 1.25″ in length, width of device 100 at widest point is about 0.75″, leg 34 is about 0.375″, and angle 21 is about 110° (angle not shown). In yet another embodiment, a plurality of legs 34 may be added to device 100 for added support based on the user's preference.

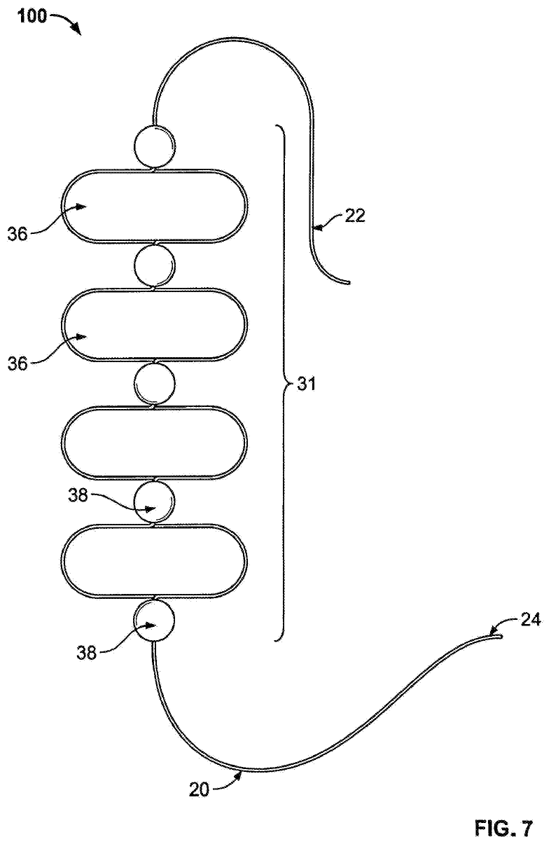

In yet another embodiment, ear shaping device 100 comprises arc 20 with angle 21, top portion 22, bottom portion 24, middle portion 31, a plurality of rings 36, and a plurality of beads 38 (see FIG. 7). In this embodiment, device 100 comprises a single-piece construction in the shape of a forward-slanting “C” and is configured to be easily placed over the user's ear. In an embodiment, middle portion 31 comprises plurality of rings 36 and plurality of bead 38 between top portion 22 and bottom portion 24 wherein plurality of rings 36 and plurality of beads 38 are permanently affixed to each other in an alternating pattern. In an embodiment, user can adjust middle portion 31 by manually stretching at least one ring 36 to the user's desired length. In an embodiment, angle 21 of arc 20 ranges from about 105° to about 120° depending on the type of the user's protruding ear (angle not shown). As depicted in FIG. 7, ring 36 and bead 38 are in an alternating pattern; however, many other configurations may be contemplated that are within the scope of the present invention.

In this embodiment, top portion 22 is about 0.375″ making device 100 about 1.25″ in length, middle portion 31 is about 0.75″, and angle 21 is about 110°. In an embodiment, top portion 22 extends along the back of the superior crus of antihelix while bottom portion 24 extends from the front of the superior crus of antihelix to the cymba allowing middle portion 31 to rest on the user's scapha and antihelix.

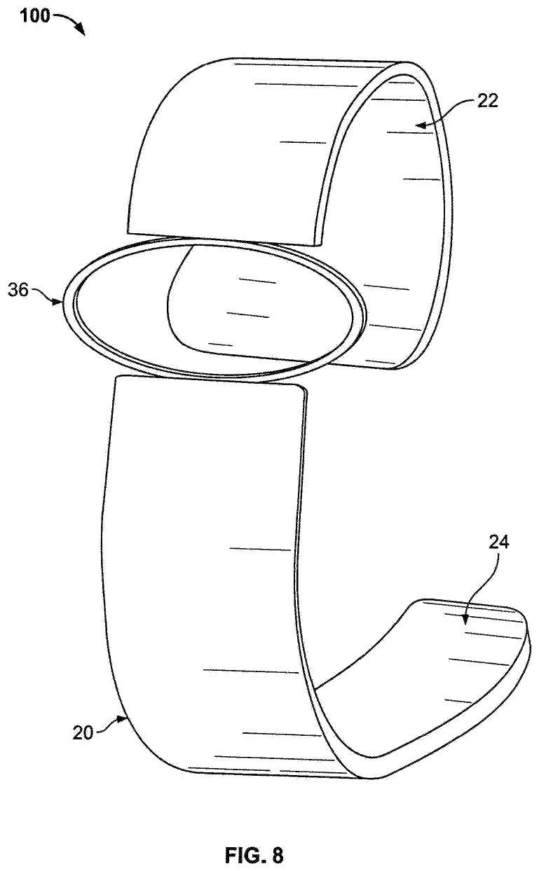

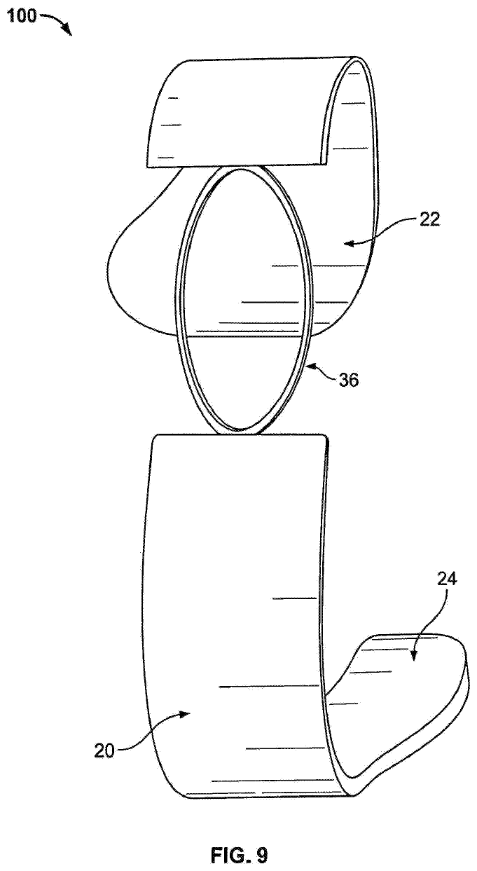

In yet another embodiment, ear shaping device 100 comprises arc 20 with angle 21, top portion 22, bottom portion 24, and ring 36 (see FIGS. 8-9). In an embodiment, device 100 comprises a single-piece construction configured to be easily placed over the user's ear wherein top portion 22 and bottom portion 24 are permanently affixed to ring 36 at opposite ends. In this embodiment, the user can adjust the length of device 100 by manually stretching ring 36 to a desired length (see FIG. 9). In one embodiment, angle 21 of arc 20 ranges from about 105° to about 120° depending on the type of the user's protruding ear (angle not shown).

In this embodiment, top portion 22 is about 0.375″ making device 100 about 1.5″ in length, ring 36 is between about 0.1875″ to 0.5″, and angle 21 is about 110°. In one embodiment, top portion 22 extends along the back of the superior crus of antihelix while bottom portion 24 extends from the front of the superior crus of antihelix to the cymba allowing middle portion 31 to rest on the user's scapha and antihelix.

In yet another embodiment, plurality of rings 36 are permanently affixed to top portion 22 and bottom portion 24 by a metal wire or similar material.

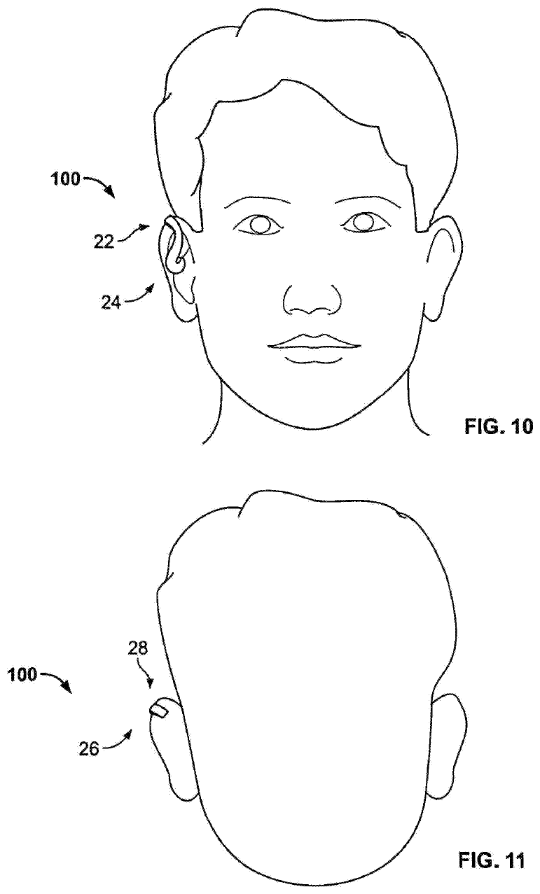

FIGS. 10 and 11 depict exemplary embodiment of ear shaping device 100 placed on a user's ear. FIG. 10 depicts ear shaping device 100 on the front of a user's ear. FIG. 11 depicts ear shaping device 100 on the back of a user's ear. In this exemplary embodiment, the user slides device 100 over the helix located at the top of the ear whereby interior surface 26 of device 100 is touching the back of the user's ear and exterior surface 28 extends behind the user's ear. In an embodiment, top portion 22 of device 100 extends over the user's helix whereby the bottom portion 24 of device 100 extends over the front of the user's ear. This embodiment is configured to allow device 100 to be easily placed over the user's ear.

Thus, specific embodiments of an ear shaping device and methods to use such device have been disclosed. It should be apparent, however, to those skilled in the art that many more modifications besides those already described are possible without departing from the inventive concepts herein. Moreover, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. An ear shaping device for the correction of protruding ears, comprising:
(a) a top and bottom portion;
(b) an interior and exterior surface; and
(c) an arc, wherein said arc is configured to have an angle, wherein said top and bottom portion are configured to curve inward towards said interior surface and said top portion has a greater curve than said bottom portion; and
wherein a user slides said ear shaping device over said user's helix and said interior surface touches the back of said user's ear.

2. The ear shaping device as recited in claim 1, wherein said ear shaping device is about 0.5 to 1.75 inches in length, about 0.375 to 0.75 inches in width, and about 0.9 to 1.5 millimeters in thickness.

3. The ear shaping device as recited in claim 1, wherein said ear shaping device comprises gold, silver, resin, plastic, any precious metal, any base metal, any reactive metal, sheet metal, wire, wire mesh, wood, or any combination thereof.

4. The ear shaping device as recited in claim 1, wherein said angle ranges from about 90 to 120 degrees and can be adjusted based on the size and specific amount of protrusion of said user's ear.

5. The ear shaping device as recited in claim 1, further comprising an interior coating or padding integrated into said ear shaping device.

6. The ear shaping device as recited in claim 1, further comprising a decorative element permanently or removably affixed to said exterior surface, wherein said decorative element comprises a gemstone or an embellishment.

7. The ear shaping device as recited in claim 6, further comprising a leg, wherein said leg is permanently affixed to said exterior or said interior surface of said top portion and said decorative element.

8. The ear shaping device as recited in claim 7, wherein said leg comprises a plurality of legs.

9. The ear shaping device as recited in claim 1, further comprising a middle portion, wherein said middle portion comprises a plurality of rings and a plurality of beads between said top portion and said bottom portion, and wherein said middle portion is adjustable by manually stretching at least one said ring to said user's desired length.

10. The ear shaping device as recited in claim 9, wherein said plurality of rings and said plurality of beads are permanently affixed to each other in an alternating pattern or any other configuration thereof.

11. The ear shaping device as recited in claim 1, further comprising a ring, wherein said top portion and said bottom portion is permanently affixed to said ring at opposite ends, and wherein length of said ear shaping device is adjustable by manually stretching said ring to desired length.

12. The ear shaping device as recited in claim 11, further comprising a plurality of rings, wherein said plurality of rings are permanently affixed to said top portion and said bottom portion by a metal wire or a similar wire forming material.

13. An ear shaping device for the correction of protruding ears, comprising:
(a) an interior and exterior surface, wherein said interior surface comprises a coating or padding;
(a) a top and bottom portion; wherein said top and said bottom portion are configured to curve inward towards said interior surface and said top portion has a greater curve than said bottom portion;
(c) an arc, wherein said arc is configured to have an angle, wherein said angle ranges from about 90 to 120 degrees and can be adjusted based on the size and specific amount of protrusion of said user's ear; and
(d) a decorative element, wherein said decorative element is permanently or removably affixed to said exterior surface, and wherein said decorative element comprises a gemstone or an embellishment; and
wherein a user slides said ear shaping device over said user's helix and said interior surface touches the back of said user's ear.

14. The ear shaping device as recited in claim 13, wherein said ear shaping device is about 0.5 to 1.75 inches in length, about 0.375 to 0.75 inches in width, and about 0.9 to 1.5 millimeters in thickness.

15. The ear shaping device as recited in claim 13, wherein said ear shaping device comprises gold, silver, resin, plastic, any precious metal, any base metal, any reactive metal, sheet metal, wire, wire mesh, wood, or any combination thereof.

16. The ear shaping device as recited in claim 13, further comprising a leg, wherein said leg is permanently affixed to said exterior or said interior surface of said top portion and said decorative element, and wherein said leg comprises a plurality of legs.

17. An ear shaping device for the correction of protruding ears, comprising:
(a) an interior and exterior surface, wherein said interior surface comprises a coating or padding;
(b) a top and bottom portion, wherein said top and said bottom portion are configured to curve inward towards said interior surface and said top portion has a greater curve than said bottom portion;
(c) an arc, wherein said arc is configured to have an angle, wherein said angle ranges from about 105 to 120 degrees and can be adjusted based on the size and specific amount of protrusion of said user's ear; and
(d) a ring, wherein said top portion and said bottom portion are permanently affixed to said ring at opposite ends, wherein said ring is adjustable by manually stretching said ring to desired length,
wherein a user slides said ear shaping device over said user's helix and said interior surface touches the back of said user's ear; and
wherein said ear shaping device is about 0.5 to 1.75 inches in length, about 0.375 to 0.75 inches in width, and about 0.9 to 1.5 millimeters in thickness.

18. The ear shaping device as recited in claim 17, wherein said ear shaping device comprises gold, silver, resin, plastic, any precious metal, any base metal, any reactive metal, sheet metal, wire, wire mesh, wood, or any combination thereof.

19. The ear shaping device as recited in claim 17, wherein said ring comprises plurality of rings permanently affixed to said top portion and said bottom portion by a metal wire or a similar wire forming material.

20. The ear shaping device as recited in claim 17, further comprising a middle portion, wherein said middle portion comprises a plurality of rings and a plurality of beads permanently affixed to each other in an alternating pattern or any other configuration thereof between said top portion and said bottom portion, and wherein said middle portion is adjustable by manually stretching at least one said ring.
